(19) United States
(12) Patent Application Publication
(10) Pub. No.: US 2016/0221897 A1
(43) Pub. Date: Aug. 4, 2016

(54) Transalkylation of Heavy Aromatic Hydrocarbon Feedstocks

(71) Applicant: ExxonMobil Chemical Patents Inc., Baytown, TX (US)

(72) Inventors: Christine N. Elia, Bridgewater, NJ (US); Wenyih F. Lai, Bridgewater, NJ (US); Shifang L. Luo, Annandale, NJ (US); Jeevan S. Abichandani, Houston, TX (US); Robert G. Tinger, Friendswood, TX (US); Gary D. Mohr, Sunset, SC (US); Xiaobo Zheng, Houston, TX (US)

(21) Appl. No.: 15/004,727

(22) Filed: Jan. 22, 2016

Related U.S. Application Data
(60) Provisional application No. 62/111,737, filed on Feb. 4, 2015.

(51) Int. Cl. C07C 6/06; C07C 4/18; B01J 8/04

(52) U.S. Cl. CPC C07C 6/06; B01J 8/04; C07C 4/18; C07C 2529/80; C07C 2529/44; C07C 2529/74; B01J 2208/027

(57) ABSTRACT

A process for producing xylene comprises contacting a first feed comprising C₉₊ aromatic hydrocarbons and hydrogen with a first catalyst composition comprising a first molecular sieve having a Constraint Index of 3 to 12 and at least one hydrogenation component. The first catalyst composition dealkylates at least part of the C₉₊ aromatic hydrocarbons containing C₂₊ alkyl groups and to saturate the resulting C₂₊ olefins to produce a second feed. The second feed is then contacted with a second catalyst composition under conditions effective to transalkylate at least part of the C₉₊ aromatic hydrocarbons in the second feed to produce a product comprising xylene. The second catalyst composition comprises a second molecular sieve having a Constraint Index less than 3 and a third molecular sieve having a Constraint Index of 3 to 12.

### CROSS-REFERENCE OF RELATED APPLICATIONS

This application claims the benefit of Provisional Application No. 62/111,737, filed Feb. 4, 2015.

### FIELD

This disclosure relates to transalkylation of heavy (C₉₊) aromatic hydrocarbon feedstocks to lighter aromatic products, especially xylenes.

### BACKGROUND

An important source of xylene in an oil refinery is catalytic reformate, which is produced by contacting a mixture of petroleum naphtha and hydrogen with a strong hydrogenation/dehydrogenation catalyst, such as platinum, on a moderately acidic support, such as a halogen-treated alumina. A C₆ to C₈ fraction is separated from the reformate and extracted with a solvent selective for aromatics or aliphatics to produce a mixture of aromatic compounds that is relatively free of aliphatics. This mixture of aromatic compounds contains benzene, toluene and xylenes (BTX), along with ethylbenzene, and can be processed in known manner to recover the xylene.

However, the quantity of xylene available from reforming is limited and so recently refineries have also focused on the production of xylene by transalkylation of heavy (C₉₊) aromatic hydrocarbons over noble metal-containing zeolite catalysts. For example, U.S. Pat. No. 5,942,651 discloses a process for the transalkylation of heavy aromatics comprising contacting a feed comprising C₉₊ aromatic hydrocarbons and toluene with a first catalyst composition comprising a molecular sieve having a constraint index ranging from 0.5 to 3, such as ZSM-12, and a hydrogenation component under transalkylation reaction conditions to produce a transalkylation reaction product comprising benzene and xylene. The transalkylation reaction product is then contacted with a second catalyst composition which comprises a molecular sieve having a constraint index ranging from 3 to 12, such as ZSM-5, and which may be in a separate bed or a separate reactor from the first catalyst composition, under conditions to remove benzene co-boilers in the product.

One problem associated with heavy aromatics transalkylation processes is catalyst aging since, as the catalyst loses activity with increasing time on stream, higher temperatures tend to be required to maintain constant conversion. When the maximum reactor temperature is reached, the catalyst needs to be replaced or regenerated, normally by oxidation. In particular, it has been found that the aging rate of existing transalkylation catalysts is strongly dependent on the presence in the feed of aromatic compounds having alkyl substituents with two or more carbon atoms, such as ethyl and propyl groups. Thus, these compounds tend to undergo disproportionation to produce C₁₀₊ coke precursors.

To address the problem of C₉₊ feeds containing high levels of ethyl and propyl substituents, U.S. Published Application No. 2009/0112034 discloses a multi-component catalyst system adapted for transalkylation of a C₉₊ aromatic feedstock with a C₆-C₇ aromatic feedstock comprising: (a) a first catalyst bed comprising a first molecular sieve having a Constraint Index in the range of 3-12 and 0.01 to 5 wt. % of at least one source of a first metal element of Groups 6-10 of the Periodic Table and (b) a second catalyst bed comprising a second molecular sieve having a Constraint Index less than 3 and 0 to 5 wt. % of at least one source of a second metal element of Groups 6-10 of the Periodic Table, wherein the weight ratio of said first catalyst to said second catalyst is in the range of 5:95 to 75:25. The first catalyst, which is optimized for dealkylation of the ethyl and propyl groups in the feed, is located in front of the second catalyst, which is optimized for transalkylation, when they are brought into contact with a C₉₊ aromatic feedstock and a C₆-C₇ aromatic feedstock in the presence of hydrogen.

U.S. Pat. No. 7,485,763 discloses a process for converting C₉₊ aromatic hydrocarbons to lighter aromatic products including xylenes, comprising the step of contacting a feed comprising C₉₊ aromatic hydrocarbons under transalkylation reaction conditions with a coextruded catalyst composition comprising the molecular sieves ZSM-12 and ZSM-5 wherein at least the ZSM-12 has a hydrogenation component associated therewith and the hydrogenation component is selected from rhenium, palladium, and mixtures thereof.

U.S. Ser. No. 62/007,556, filed June 4, 2014 discloses a process for producing xylene comprising contacting a first feed comprising C₉₊ aromatic hydrocarbons, at least one C₆-C₇ aromatic hydrocarbon and hydrogen with a first catalyst composition to dealkylate at least part of the C₉₊ aromatic hydrocarbons containing C₂₊ alkyl groups and to saturate the resulting C₂₊ olefins to produce a second feed. The second feed is then contacted with a second catalyst composition under conditions effective to transalkylate at least part of the C₉₊ aromatic hydrocarbons with at least part of the C₆-C₇ aromatic hydrocarbon to produce a first product comprising xylene. Each of the first and second catalyst compositions is substantially free of amorphous alumina.

However, despite these and other advances, there remains a need to further improve the catalysts employed in C₉₊ aromatic transalkylation processes.

Other references of interest include: U.S. Pat. Nos. 8,481,443, 7,485,763, 5,271,920, and 4,900,529; U.S. Publication Nos. 2011/0118520, 2005/0065017, 2010/0094068, 2010/0093520, 2012/0244049, 2010/0029467, 2005/0215838, 2008/0035525; and EP Patent Nos. 1,586,376, 1,655,277, and 141514.

### SUMMARY

According to the present disclosure, it has now been found that, in the production of xylene from C₉₊ aromatic hydrocarbons, improvements in BTX yield and ethyl-aromatic conversion activity can be achieved by using a multi-component catalyst system comprising (a) a first catalyst composition comprising a first molecular sieve having a Constraint Index of 3 to 12 and (b) a second catalyst composition comprising both a second molecular sieve having a Constraint Index less than 3 and a third molecular sieve having a Constraint Index of 3 to 12.

Thus, the present disclosure relates to a process for producing xylene from C₉₊ aromatic hydrocarbons, the process comprising:
(a) contacting a first feed comprising C₉₊ aromatic hydrocarbons and hydrogen with a first catalyst composition under conditions effective to dealkylate at least part of the C₉₊ aromatic hydrocarbons containing C₂₊ alkyl groups and to saturate the resulting C₂₊ olefins to produce a second feed, wherein the first catalyst composition comprises a first molecular sieve having a Constraint Index of 3 to 12 and at least one hydrogenation component; and
(b) contacting the second feed with a second catalyst composition under conditions effective to transalkylate at least part of the C₉₊ aromatic hydrocarbons in the second feed to produce a first product comprising xylene, wherein the second catalyst composition comprises a second molecular sieve having a Constraint Index less than 3 and a third molecular sieve having a Constraint Index of 3 to 12.

In embodiments, the first molecular sieve comprises ZSM-5, the second molecular sieve comprises ZSM-12 and/or mordenite and the third molecular sieve comprises ZSM-5.

In one embodiment, the second molecular sieve and the third molecular sieve are contained in a single catalyst particle.

In one embodiment, the at least one hydrogenation component of the first catalyst composition comprises at least one metal or compound thereof selected from Groups 6-10 of the Periodic Table and tin or a tin compound.

In one embodiment, the second catalyst composition further comprises at least one metal or compound thereof selected from Groups 6-10 of the Periodic Table and tin or a tin compound.

In embodiments, the process further comprises:
(c) contacting at least part of the first product with a third catalyst composition under conditions effective to remove benzene coboilers in the first product and produce a second product, wherein the third catalyst composition comprises a fourth molecular sieve having a Constraint Index of 3 to 12.

In a further aspect, the present disclosure relates to a catalyst system for transalkylating a feed comprising C₉₊ aromatic hydrocarbons to produce xylene, the catalyst system comprising:
(i) a first catalyst composition comprising a first molecular sieve having a Constraint Index of 3 to 12 and a hydrogenation component;
(ii) a second catalyst composition comprising a second molecular sieve having a Constraint Index less than 3 and a third molecular sieve having a Constraint Index of 3 to 12, the second catalyst composition being located downstream of the first catalyst composition when the catalyst system is contacted with the feed; and
(iii) a third catalyst composition comprising a fourth molecular sieve having a Constraint Index of 3 to 12, the third catalyst composition being located downstream of the second catalyst composition when the catalyst system is contacted with the feed.

### BRIEF DESCRIPTION OF THE DRAWINGS

FIG. 1 is a graph of ethyl aromatic conversion against temperature for the heavy aromatic conversion processes of Examples 1 to 4.

FIG. 2 is a graph of benzene/toluene/xylene (BTX) yield against temperature for the heavy aromatic conversion processes of Examples 1 to 4.

FIG. 3 is a graph of C₉₊ conversion against temperature for the heavy aromatic conversion processes of Examples 4 to 6.

FIG. 4 is a graph of ethyl aromatic conversion against temperature for the heavy aromatic conversion processes of Examples 4 to 6.

FIG. 5 is a graph of trimethylbenzene conversion against temperature for the heavy aromatic conversion processes of Examples 4 to 6.

FIG. 6 is a graph of benzene/toluene/xylene (BTX) yield against temperature for the heavy aromatic conversion processes of Examples 4 to 6.

FIG. 7 is a graph of C₉₊ conversion against temperature for the heavy aromatic conversion processes of Examples 5 and 7.

FIG. 8 is a graph of ethyl aromatic conversion against temperature for the heavy aromatic conversion processes of Examples 5 and 7.

FIG. 9 is a graph of benzene/toluene/xylene (BTX) yield against temperature for the heavy aromatic conversion processes of Examples 5 and 7.

### DETAILED DESCRIPTION OF THE EMBODIMENTS

As used herein, the numbering scheme for the Periodic Table Groups is as described in Chemical and Engineering News, 63(5), 27 (1985).

The term “aromatic” is used herein in accordance with its art-recognized scope which includes alkyl substituted and unsubstituted mono- and polynuclear compounds. Preferred C₉₊ aromatic hydrocarbons for use in the present process are alkyl substituted benzenes.

The term “ethyl-aromatic compounds” means aromatic compounds having an ethyl group attached to the aromatic ring. The term “propyl-aromatic compounds” means aromatic compounds having a propyl group attached to the aromatic ring.

The term “Cₙ” hydrocarbon or aromatic as used herein means a hydrocarbon or aromatic compound having n number of carbon atom(s) per molecule. The term “Cₙ₊” hydrocarbon or aromatic means hydrocarbon or aromatic compound having n or more than n carbon atom(s) per molecule. The term “Cₙ” hydrocarbon or aromatic means a hydrocarbon or aromatic compound having less than n carbon atom(s) per molecule.

The term “benzene coboiler(s)” means impurities that have a boiling point close to the boiling point of benzene (typically plus or minus 10° C.) as described at page 393 of Aromatic Hydrocarbons—Advances in Research and Treatment, 2013 Edition, Acton, Q. A. Ed., Scholarly Editions, Atlanta, Ga. (2013).

The term “substantially free” when used in relation to a specific component of a catalyst composition means that the catalyst composition contains less than 1 wt. %, preferably less than 0.15 wt. %, of that component.

The overall surface area (also referred to as total surface area) of a molecular sieve may be measured by the Brunauer-Emmett-Teller (BET) method using adsorption-desorption of nitrogen (temperature of liquid nitrogen, 77 K). The internal surface area may be calculated using t-plot of the Brunauer-Emmett-Teller (BET) measurement. The external surface area is calculated by subtracting the internal surface area from the overall surface area measured by the Brunauer-Emmett-Teller (BET) measurement.

Particle size is measured by averaging the size of multiple particles as shown in SEM images obtained on a HITACHI S4800 Field Emission Scanning Electron Microscope (SEM). The particle size is measured by averaging the size of multiple particles as shown in the SEM. The same method is used for crystal size. Transmission Electron Microscopy may also be used, but in event of conflict between SEM and TEM, SEM shall control.

Aromatic ring-loss, as used herein, is calculated by the following formula:

Aromatic Ring loss (%)=(1−total moles of aromatic compounds in product/total moles of aromatic compounds in feed)*100.

Described herein is a process for producing xylenes from C₉₊ aromatic hydrocarbons using a series-connected multiple bed catalyst system. In most embodiments, the process employs a first catalyst bed comprising a first catalyst composition selective for the dealkylation of ethyl-aromatic compounds and propyl-aromatic compounds in the C₉₊ aromatic hydrocarbon feed. Downstream of the first catalyst bed is a second catalyst bed comprising a second catalyst composition effective to transalkylate C₉₊ aromatic hydrocarbons, optionally with cofed benzene and/or toluene to produce xylenes. In most embodiments, the xylene-containing product of the transalkylation step is then passed to a third catalyst bed which is located downstream of the second catalyst bed and which comprises a third catalyst composition effective to remove benzene co-boilers in the product. In some embodiments, the third catalyst bed is omitted and the process employs the first and second catalysts beds only. Preferably, however, the process employs the first, second and third catalyst beds.

Each catalyst bed can be housed in a separate reactor or, where desired, two or more of the catalysts beds can be accommodated in the same reactor. For example, the first, second and optionally the third catalyst beds can be stacked one on top of the other in a single reactor. In all situations, the first catalyst is not mixed with the second catalyst and the hydrocarbon feedstocks and hydrogen are contacted with the first catalyst prior to contacting the second catalyst.

### Feedstock

The aromatic feed used in the present process comprises one or more aromatic hydrocarbons containing at least 9 carbon atoms. Specific C₉₊ aromatic compounds found in a typical feed include mesitylene (1,3,5-trimethylbenzene), durene (1,2,4,5-tetramethylbenzene), hemimellitene (1,2,4-trimethylbenzene), pseudocumene (1,2,4-trimethylbenzene), 1,2-methylethylbenzene, 1,3 -methylethylbenzene, 1,4-methylethylbenzene, propyl-substituted benzenes, butyl-substituted benzenes, and dimethylethylbenzenes. Suitable sources of the C₉₊ aromatics are any C₉₊ fraction from any refinery process that is rich in aromatics. This aromatics fraction contains a substantial proportion of C₉₊ aromatics, e.g., at least 80 wt. % C₉₊ aromatics, wherein preferably at least 80 wt. %, and more preferably more than 90 wt. %, of the hydrocarbons will range from C₉ to C₁₂. Typical refinery fractions which may be useful include catalytic reformate, FCC (fluid catalyst cracking) naphtha or TCC (temperature catalyst cracking) naphtha.

The feed to the process may also include one or more C₆₋₇ aromatics, such as benzene and/or toluene. In one practical embodiment, the feed to the transalkylation reactor comprises C₉₊ aromatics hydrocarbons and toluene. The feed may also include recycled/unreacted toluene and C₉₊ aromatic feedstock that is obtained by distillation of the effluent product of the transalkylation reaction itself. Typically, toluene constitutes from 0 to 90 wt. %, such as from 10 to 70 wt. % of the entire feed, whereas the C₉₊ aromatics component constitutes from 10 to 100 wt. %, such as from 30 to 85 wt. % of the entire feed to the process.

The feed to the process will also normally include hydrogen to saturate the C₂₊ olefins generated by the dealkylation reactions occurring in the optional first catalyst bed.

### First Catalyst Bed

The first catalyst bed employed in the present catalyst system contains a first catalyst composition comprising a first molecular sieve having a Constraint Index in the range of about 3 to about 12 and at least one hydrogenation component.

Constraint Index is a convenient measure of the extent to which an aluminosilicate or other molecular sieve provides controlled access to molecules of varying sizes to its internal structure. For example, molecular sieves which provide a highly restricted access to and egress from its internal structure have a high value for the Constraint Index. Molecular sieves of this kind usually have pores of small diameter, e.g., less than 5 Angstroms. On the other hand, molecular sieves which provide relatively free access to their internal pore structure have a low value for the constraint index, and usually pores of large size. The method by which constraint index is determined is described fully in U.S. Pat. No. 4,016,218, which is incorporated herein by reference for the details of the method.

Suitable molecular sieves for use in the first catalyst composition comprise at least one of ZSM-5, ZSM-11, ZSM-22, ZSM-23, ZSM-35, ZSM-48, ZSM-57 and ZSM-58. ZSM-5 is described in detail in U.S. Pat. No. 3,702,886 and Re. 29,948. ZSM-11 is described in detail in U.S. Pat. No 3,709,979. ZSM-22 is described in U.S. Pat. Nos. 4,556,477 and 5,336,478. ZSM-23 is described in U.S. Pat. No. 4,076,842. ZSM-35 is described in U.S. Pat. No. 4,016,245. ZSM-48 is more particularly described in U.S. Pat. Nos. 4,234,231 and 4,375,573. ZSM-57 is described in U.S. Pat. No. 4,873,067. ZSM-58 is described in U.S. Pat. No. 4,698,217.

In one preferred embodiment, the first molecular sieve comprises ZSM-5 and especially ZSM-5 having an average crystal size (primary particle) of less than 0.1 micron, for example such that the ZSM-5 crystals have an external surface area in excess of 50 m²/g, preferably in excess of 70 m²/g, as determined by the t-plot method for nitrogen physisorption. Suitable ZSM-5 compositions are disclosed in PCT/US2013/071456, filed Nov. 22, 2013 (which claims priority to U.S. Ser. No. 61/740,908, filed Dec. 21, 2012) and PCT/US2013/071446, filed Nov. 22, 2013 (which claims priority to U.S. Ser. No. 61/740,917, filed Dec. 21, 2012, the entire contents of which are incorporated herein by reference).

Conveniently, the first molecular sieve has an alpha value in the range of about 100 to about 1500, such as about 150 to about 1000, for example about 150 to about 600. Alpha value is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395.

Generally, the first molecular sieve is an aluminosilicate having a silica to alumina molar ratio of less than 1000, typically from about 10 to about 100.

Typically, the first catalyst composition comprises at least 1 wt. %, preferably at least 10 wt. %, more preferably at least 25 wt. %, and most preferably at least 50 wt. %, of the first molecular sieve. In one embodiment, the first catalyst composition comprises from 55 to 80 wt. % of the first molecular sieve.

In addition to a molecular sieve having a Constraint Index in the range of about 3 to about 12, the first catalyst composition comprises at least one hydrogenation component, such as at least one metal or compound thereof of Groups 6 to 12 of the Periodic Table of the Elements. Suitable hydrogenation components include platinum, palladium, iridium, rhenium and mixtures and compounds thereof, preferably platinum, rhenium and compounds thereof. In some embodiments, the first catalyst composition comprises two or more hydrogenation components including a first metal or compound thereof selected from platinum, palladium, iridium, rhenium and mixtures thereof and a second metal or compound chosen so as to lower the benzene saturation activity of the first metal. Examples of suitable second metals include at least one of copper, silver, gold, ruthenium, iron, tungsten, molybdenum, cobalt, nickel, tin and zinc. Conveniently, the first metal is present in the first catalyst in an amount from 0.001 to 1 wt. %, such as from 0.01 to 0.1 wt. %, of the first catalyst and the second metal is present in the first catalyst in amount from 0.001 to 10 wt. %, 0.1 to 1 wt. %, of the first catalyst.

In some embodiments, the first metal comprises platinum and/or rhenium and the second metal comprises copper and/or tin. In one preferred embodiment, the first metal comprises platinum and the second metal comprises tin, desirably at a molar ratio of platinum to tin from 0.1:1 to 1:1, such as from 0.2:1 to 0.4:1.

The hydrogenation component can be incorporated into the first catalyst composition by any known method, including by ion exchange into the composition to the extent a Group 13 element, e.g., aluminum, is in the molecular sieve structure, by impregnation (such as by incipient wetness) or by mixing with the molecular sieve and/or binder. In some embodiments, ion exchange may be preferred. After incorporation of the hydrogenation component(s), the catalyst composition is usually dried by heating at a temperature of 65° C. to 160° C., typically 110° C. to 143° C., for at least 1 minute and generally not longer than 24 hours, at pressures ranging from 100 to 200 kPa-a. Thereafter, the catalyst composition may be calcined in a stream of dry gas, such as air or nitrogen, at temperatures of from 260° C. to 650° C. for 1 to 20 hours. Calcination is typically conducted at pressures ranging from 100 to 300 kPa-a. In some embodiments, the hydrogenation components(s) are combined with the first molecular sieve before formation of the final catalyst particles.

The first catalyst composition may be self-bound (that is without a separate binder) or may also comprise a binder or matrix material that is resistant to the temperatures and other conditions employed in the present process. Suitable binder materials include silica, clays and metal oxides, such as alumina, zirconia, titania, silica-alumina, silica-magnesia, silica-zirconia, silica-thoria, silica-beryllia, silica-titania as well as ternary compositions, such as silica-alumina-thoria, silica-alumina-zirconia, silica-alumina-magnesia, and silica-magnesia-zirconia. Where a binder or matrix material is present, it is desirably substantially free of amorphous alumina, since it is found that the exclusion of a binder containing amorphous alumina reduces external catalytic sites for coke production and hence increases catalyst cycle length. One preferred binder material for the first catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the first molecular sieve.

Where the first catalyst composition contains a binder or matrix material, the latter may be present in an amount ranging from 5 to 95 wt. %, and typically from 10 to 60 wt. %, of the total catalyst composition.

The first catalyst composition may be extruded into particles of any desired shape before being loaded into the first catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to less than 200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

Prior to use, at least the molecular sieve component of the first catalyst composition may be subjected to steam treatment. For example, extruded particles of the molecular sieve, optionally together with a binder, may be subjected to steam treatment prior to incorporation of the hydrogenation component(s). Alternatively, steaming can be conducted after the incorporation of the hydrogenation component(s). Suitable conditions for the steam treatment process comprise contacting the molecular sieve component with from 5 to 100% steam at a temperature from 260° C. to 650° C. for at least one hour, specifically 1 to 20 hours, at a pressure of 100 to 2590 kPa-a.

In an embodiment, the first catalyst composition may be treated to reduce the aromatics hydrogenation activity of the catalyst. One method for minimizing the aromatics hydrogenation activity of a catalyst composition is by exposing it to a compound containing an element selected from Group 15 or 16 of the Periodic Table of the Elements, preferably N, P, S, O. The Group 16 element specifically contemplated is sulfur. A specifically contemplated Group 15 element is nitrogen. Effective treatment is accomplished by contacting the catalyst with a source of sulfur at a temperature ranging from about 200° C. to 480° C. The source of sulfur can be contacted with the catalyst via a carrier gas, typically, an inert gas such as hydrogen or nitrogen. In a useful embodiment, the source of sulfur is typically hydrogen sulfide.

The first catalyst composition can also be treated in situ. For example, a source of sulfur is contacted with the catalyst composition by adding it to the hydrocarbon feedstream in a concentration ranging from about 50 ppmw sulfur to about 10,000 ppmw sulfur. Any sulfur compound that will decompose to form H₂S and a light hydrocarbon at about 490° C. or less will suffice. Typical examples of useful sources of sulfur include carbon disulfide and alkylsulfides such as methylsulfide, dimethylsulfide, dimethyldisulfide, diethylsulfide and dibutyl sulfide. Sulfur treatment can be considered sufficient when sulfur breakthrough occurs; that is, when sulfur appears in the liquid product.

Typically, sulfur treatment is initiated by incorporating a source of sulfur into the feed and continuing sulfur treatment for a few days, typically, up to 10 days, more specifically, from one to five days. The sulfur treatment can be monitored by measuring the concentration of sulfur in the product off gas. During this treatment, the sulfur concentration in the off gas should range from about 20 to about 500 ppmw sulfur, preferably about 30 to 250 ppmw.

Continuously cofeeding a source of sulfur has been found to maintain reduced aromatics hydrogenation activity. The catalyst can be contacted with sulfur during service by cofeeding sulfur to the reactor in varied amounts via the hydrogen stream entering the reactor or the hydrocarbon feedstock. The sulfur can be continuously added to the feedstock throughout the process cycle or the sulfur can be intermittently continuously added in which this sulfur is cofed continuously for a period of time, discontinued, then cofed again.

In embodiments, the first catalyst composition comprises from 10 wt. % to 50 wt. %, for example from 15 wt. % to 35 wt. %, of the total weight of the first, second and third catalyst compositions.

In operation, the first catalyst bed is maintained under conditions effective to dealkylate aromatic hydrocarbons containing C₂₊ alkyl groups in the heavy aromatic feedstock and to saturate the resulting C₂₊ olefins. Suitable conditions for operation of the first catalyst bed comprise a temperature in the range of about 100° C. to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 2 to about 20 hr⁻¹.

### Second Catalyst Bed

The second catalyst bed contains a second catalyst composition comprising a second molecular sieve having a Constraint Index less than 3 and a third molecular sieve having a Constraint Index of 3 to 12.

Examples of molecular sieves having a Constraint Index less than 3 suitable for use as the second molecular sieve comprise at least one of zeolite beta, zeolite Y, Ultrastable Y (USY), Dealuminized Y (Deal Y), mordenite, NU-87, ZSM-3, ZSM-4 (Mazzite), ZSM-12, ZSM-18, MCM-22, PSH-3, SSZ-25, MCM-36, MCM-49, MCM-56, EMM-10, EMM-10-P and ZSM-20. Zeolite ZSM-4 is described in U.S. Pat. No. 3,923,636. Zeolite ZSM-12 is described in U.S. Pat. No. 3,832,449. Zeolite ZSM-20 is described in U.S. Pat. No. 3,972,983. Zeolite Beta is described in U.S. Pat. No. 3,308,069, and Re. No. 28,341. Low sodium Ultrastable Y molecular sieve (USY) is described in U.S. Pat. Nos. 3,293,192 and 3,449,070. Dealuminized Y zeolite (Deal Y) may be prepared by the method found in U.S. Pat. No. 3,442,795. Zeolite UHP-Y is described in U.S. Pat. No. 4,401,556. Rare earth exchanged Y (REY) is described in U.S. Pat. No. 3,524,820. Mordenite is a naturally occurring material but is also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104. MCM-22 is described in U.S. Pat. No. 4,954,325. PSH-3 is described in U.S. Pat. No. 4,439,409. SSZ-25 is described in U.S. Pat. No. 4,826,667. MCM-36 is described in U.S. Pat. No. 5,250,277. MCM-49 is described in U.S. Pat. No. 5,236,575. MCM-56 is described in U.S. Pat. No. 5,362,697.

In one preferred example, the second molecular sieve comprises ZSM-12 and especially ZSM-12 having an average crystal size (primary particle) of less than 0.1 micron, such as about 0.05 micron or less.

Examples of molecular sieves having a Constraint Index of 3 to 12 suitable for use as the third molecular sieve comprise at least one of ZSM-5, ZSM-11, ZSM-22, ZSM-23, ZSM-35, ZSM-48, ZSM-57, ZSM-58, and the MCM-22 family of zeolites. ZSM-5 is described in detail in U.S. Pat. No. 3,702,886 and Re. 29,948. ZSM-11 is described in detail in U.S. Pat. No 3,709,979. ZSM-22 is described in U.S. Pat. Nos. 4,556,477 and 5,336,478. ZSM-23 is described in U.S. Pat. No. 4,076,842. ZSM-35 is described in U.S. Pat. No. 4,016,245. ZSM-48 is more particularly described in U.S. Pat. Nos. 4,234,231 and 4,375,573. ZSM-57 is described in U.S. Pat. No. 4,873,067. ZSM-58 is described in U.S. Pat. No. 4,698,217. The third molecular sieve may be the same as, or different from, the first molecular sieve.

In one preferred embodiment, the third molecular sieve comprises ZSM-5 and especially ZSM-5 having an average crystal size (primary particle) of less than 0.1 micron and an external surface area in excess of 50 m²/g, preferably in excess of 70 m²/g, as determined by the t-plot method for nitrogen physisorption (see foregoing description of the first molecular sieve).

The second and third molecular sieves can be incorporated in the second catalyst composition as a mixture of separate particles but, more preferably, are contained in the same particle, for example by co-extrusion of the second and third molecular sieves alone or in combination with a binder or matrix material.

Typically, the second catalyst composition comprises at least 1 wt. %, preferably at least 10 wt. %, more preferably at least 25 wt. %, and most preferably at least 50 wt. %, of the combination of the second and third molecular sieves. In one embodiment, the second catalyst composition comprises from 55 to 80 wt. % of the combination of the second and third molecular sieves. In such a case, the combination typically comprises from 50 to 85 wt. % of second molecular sieve having a Constraint Index less than 3 and from 15 to 50 wt. % of the third molecular sieve having a Constraint Index in the range of 3 to 12, based upon the weight of the combination of the first and second molecular sieves.

In addition to the molecular sieves as described above, the second catalyst composition optionally comprises at least one hydrogenation component, such as at least one metal or compound thereof of Groups 6 to 12 of the Periodic Table of the Elements. Suitable hydrogenation components include platinum, palladium, iridium, rhenium and mixtures and compounds thereof, preferably platinum, rhenium and compounds thereof. In some embodiments, the second catalyst composition comprises two or more hydrogenation components including a first metal or compound thereof selected from platinum, palladium, iridium, rhenium and mixtures thereof and a second metal or compound chosen so as to lower the benzene saturation activity of the first metal. Examples of suitable second metals include at least one of copper, silver, gold, ruthenium, iron, tungsten, molybdenum, cobalt, nickel, tin and zinc. Conveniently, the first metal is present in the second catalyst composition in an amount from 0.001 to 1 wt. %, such as from 0.01 to 0.1 wt. %, of the second catalyst composition and the second metal is present in the second catalyst composition in amount from 0.001 to 10 wt. %, 0.1 to 1 wt. %, of the second catalyst composition.

In some embodiments, the first metal comprises platinum and/or rhenium and the second metal comprises copper and/or tin. In one preferred embodiment, the first metal comprises platinum and the second metal comprises tin, desirably at a molar ratio of platinum to tin from 0.1:1 to 1:1, such as from 0.2:1 to 0.4:1.

The hydrogenation component can be incorporated into the second catalyst composition by any known method, including by ion exchange into the composition to the extent a Group 13 element, e.g., aluminum, is in the molecular sieve structure, by impregnation (such as by incipient wetness) or by mixing with the molecular sieve and/or binder. In some embodiments, ion exchange may be preferred. After incorporation of the hydrogenation component(s), the catalyst composition is usually dried by heating at a temperature of 65° C. to 160° C., typically 110° C. to 143° C., for at least 1 minute and generally not longer than 24 hours, at pressures ranging from 100 to 200 kPa-a. Thereafter, the catalyst composition may be calcined in a stream of dry gas, such as air or nitrogen, at temperatures of from 260° C. to 650° C. for 1 to 20 hours. Calcination is typically conducted at pressures ranging from 100 to 300 kPa-a. In some embodiments, the hydrogenation components(s) are combined with the one or both of the second and third molecular sieves before formation of the final catalyst particles.

The second catalyst composition may be self-bound (that is without a separate binder) or may also comprise a binder or matrix material that is resistant to the temperatures and other conditions employed in the present process. Where such a binder or matrix material is present, it is preferably substantially free of amorphous alumina, since it is found that the exclusion of a binder containing amorphous alumina reduces external catalytic sites for coke production and hence increases catalyst cycle length. One preferred binder material for the second catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the third molecular sieve. In one preferred embodiment, each of the first and second catalyst compositions includes a silica binder.

The second catalyst composition may be extruded into particles of any desired shape before being loaded into the second catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to less than 200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

Prior to use, at least the molecular sieve components of the second catalyst composition may be subjected to steam treatment. For example, extruded particles of the second and third molecular sieves, optionally together with a binder, may be subjected to steam treatment prior to incorporation of the hydrogenation component(s). Alternatively, steaming can be conducted after the incorporation of the hydrogenation component(s). Suitable conditions for the steam treatment process comprise contacting the molecular sieve components with from 5 to 100% steam at a temperature from 260° C. to 650° C. for at least one hour, specifically 1 to 20 hours, at a pressure of 100 to 2590 kPa-a.

In an embodiment, the second catalyst composition may be treated to reduce the aromatics hydrogenation activity of the catalyst. One method for minimizing the aromatics hydrogenation activity of a catalyst composition is by exposing it to a compound containing an element selected from Group 15 or 16 of the Periodic Table of the Elements, preferably N, P, S, and O. The Group 16 element specifically contemplated is sulfur. A specifically contemplated Group 15 element is nitrogen. Effective treatment is accomplished by contacting the catalyst with a source of sulfur at a temperature ranging from about 200° to 480° C. The source of sulfur can be contacted with the catalyst via a carrier gas, typically, an inert gas such as hydrogen or nitrogen. In a useful embodiment, the source of sulfur is typically hydrogen sulfide.

The second catalyst composition can also be treated in situ. For example, a source of sulfur is contacted with the catalyst composition by adding it to the hydrocarbon feedstream in a concentration ranging from about 50 ppmw sulfur to about 10,000 ppmw sulfur. Any sulfur compound that will decompose to form H₂S and a light hydrocarbon at about 490° C. or less will suffice. Typical examples of useful sources of sulfur include carbon disulfide and alkylsulfides such as methylsulfide, dimethylsulfide, dimethyldisulfide, diethylsulfide and dibutyl sulfide. Sulfur treatment can be considered sufficient when sulfur breakthrough occurs; that is, when sulfur appears in the liquid product.

Typically, sulfur treatment is initiated by incorporating a source of sulfur into the feed and continuing sulfur treatment for a few days, typically, up to 10 days, more specifically, from one to five days. The sulfur treatment can be monitored by measuring the concentration of sulfur in the product off gas. During this treatment, the sulfur concentration in the off gas should range from about 20 to about 500 ppmw sulfur, preferably about 30 to 250 ppmw.

Continuously cofeeding a source of sulfur has been found to maintain reduced aromatics hydrogenation activity. The catalyst can be contacted with sulfur during service by cofeeding sulfur to the reactor in varied amounts via the hydrogen stream entering the reactor or the hydrocarbon feedstock. The sulfur can be continuously added to the feedstock throughout the process cycle or the sulfur can be intermittently continuously added in which this sulfur is cofed continuously for a period of time, discontinued, then cofed again.

In embodiments, the second catalyst composition comprises from 30 wt. % to 90 wt. %, for example from 50 wt. % to 75 wt. % of the total weight of the first, second and third catalyst compositions.

In operation, the second catalyst bed is maintained under conditions effective to transalkylate C₉₊ aromatic hydrocarbons with at least one C₆-C₇ aromatic hydrocarbon, such as benzene or toluene (which may be present in the product from the first catalyst bed or fed to the second catalyst bed). Suitable conditions for operation of the second catalyst bed comprise a temperature in the range of about 100° C. to about 800° C., preferably about 300° C. to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 1 to about 10 hr⁻¹.

### Third Catalyst Bed

When present, the third catalyst bed employed in the present catalyst system contains a third catalyst composition comprising a fourth molecular sieve having a Constraint Index in the range of about 3 to about 12. Suitable molecular sieves for use in the third catalyst composition comprise at least one of ZSM-5, ZSM-11, ZSM-22, ZSM-23, ZSM-35, ZSM-48, ZSM-57 and ZSM-58, with ZSM-5 being preferred.

In one preferred embodiment, the fourth molecular sieve comprises ZSM-5 and especially ZSM-5 having an average crystal size (primary particle) of less than 0.1 micron, for example such that the ZSM-5 crystals have an external surface area in excess of 50 m²/g, preferably in excess of 70 m²/g, as determined by the t-plot method for nitrogen physisorption (see foregoing description of the first molecular sieve).

Conveniently, the fourth molecular sieve has an alpha value in the range of about 100 to about 1500, such as about 150 to about 1000, for example about 150 to about 600.

The third catalyst composition may be self-bound (that is without a separate binder) or may also comprise a binder or matrix material that is resistant to the temperatures and other conditions employed in the present process. Where such a binder or matrix material is present, it is preferably substantially free of amorphous alumina, since it is found that the exclusion of a binder containing amorphous alumina reduces external catalytic sites for coke production and hence increases catalyst cycle length. One preferred binder material for the third catalyst composition comprises silica since extrusion with silica ensures that the catalyst has high mesoporosity and hence high activity. Alternatively, the binder or matrix material may be a crystalline molecular sieve material, which may be isostructural with, or have a different structure than, the third molecular sieve. In one preferred embodiment, each of the first, second and third catalyst compositions includes a silica binder.

Where the third catalyst composition contains a binder or matrix material, the latter may be present in an amount ranging from 5 to 95 wt. %, and typically from 10 to 60 wt. %, of the total catalyst composition.

The third catalyst composition may be extruded into particles of any desired shape before being loaded into the first catalyst bed. In some embodiments, it may be desirable to control the shape and size of the catalyst particles so as to maximize the external surface area of the catalyst. For example, it may be desirable to control the catalyst particle configuration such that the particles have a surface to volume ratio of about 80 to less than 200 inch⁻¹, preferably about 100 to 150 inch⁻¹. Suitable particle configurations for achieving such a surface to volume ratio include grooved cylindrical extrudates and hollow or solid polylobal extrudates, such as quadrulobal extrudates.

Prior to use, at least the molecular sieve component of the third catalyst composition may be subjected to steam treatment. For example, extruded particles of the molecular sieve, optionally together with a binder, may be subjected to steam treatment prior to incorporation of the hydrogenation component(s). Suitable conditions for the steam treatment process comprise contacting the molecular sieve component with from 5 to 100% steam at a temperature from 260° C. to 650° C. for at least one hour, specifically 1 to 20 hours, at a pressure of 100 to 2590 kPa-a.

In embodiments, the third catalyst composition comprises up to 25 wt. %, for example from 5 wt. % to 25 wt. %, such as from 5 wt. % to 15 wt. % of the total weight of the first, second and third catalyst compositions.

In operation, the third catalyst bed is maintained under conditions effective to crack non-aromatic cyclic hydrocarbons, such as benzene coboilers, in the effluent from the second catalyst bed. Examples of benzene coboilers include cyclohexane, methylcyclopentane, 2-methylhexane, 3-methylhexane, 2,3-dimethylpentane, 2,4-dimethylpentane, and dimethylcyclopentane. Suitable conditions for operation of the third catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 1 to about 50 hr⁻¹.

The invention will now be more particularly described with reference to the following non-limiting Examples and the accompanying drawings.

In the Examples, a variety of different catalyst systems were used to dealkylate/transalkylate a feed composed of 85 wt. % of a heavy aromatic feedstock and 15 wt. % toluene and having the composition given in Table 1 or Table 2.

TABLE 1

| Component | |
|---|---|
| weight % | % |
| BENZENE | 0.0 |
| TOLUENE | 15.4 |
| ETHYLBENZENE | 0.0 |
| O-XYLENE | 0.0 |
| M-XYLENE | 0.0 |
| OTHER C9 PARAFFINS | 0.0 |
| NC3BENZENE | 3.2 |
| IC3BENZENE | 1.0 |
| 1M2ETBENZENE | 5.6 |
| 1M3ETBENZENE | 13.8 |
| 1M4ETBENZENE | 6.8 |
| 123TMBENZENE | 5.7 |
| 124TMBENZENE | 28.6 |
| 135TMBENZENE | 8.3 |
| INDANE | 0.7 |
| OTHER C10 PARAFFINS | 1.3 |
| 1M3NC3BENZENE | 1.0 |
| 1M4NC3BENZENE | 0.4 |
| 1M3IC3BENZENE | 0.5 |
| 1M4IC3BENZENE | 0.1 |
| 12DETBENZENE | 0.1 |
| 13DETBENZENE | 0.6 |
| 14DETBENZENE | 0.2 |
| 12DM3ETBENZENE | 0.2 |
| 12DM4ETBENZENE | 1.5 |
| 13DM2ETBENZENE | 0.1 |
| 13DM4ETBENZENE | 0.9 |
| 13DM5ETBENZENE | 1.8 |
| 14DM2ETBENZENE | |
| 1234TMBENZENE | 0.1 |
| 1235TMBENZENE | 0.8 |
| 1245TMBENZENE | 0.6 |
| NAPHTHALENE | 0.1 |
| M-INDANES | 0.1 |
| OTHER C10 AROMATICS | 0.3 |
| 1M-NAPHTHALENE | |
| 2M-NAPHTHALENE | 0.0 |
| OTHER C11 AROMATICS | 0.0 |
| Total | 100.00 |

TABLE 2

| Component | |
|---|---|
| weight % | % |
| BENZENE | 0.01 |
| TOLUENE | 15.07 |
| ETHYLBENZENE | 0.00 |
| O-XYLENE | 0.12 |
| M-XYLENE | 0.00 |
| OTHER C9 PARAFFINS | 0.11 |
| NC3BENZENE | 2.13 |
| IC3BENZENE | 0.52 |
| 1M2ETBENZENE | 4.66 |
| 1M3ETBENZENE | 11.45 |
| 1M4ETBENZENE | 4.92 |
| 123TMBENZENE | 6.45 |
| 124TMBENZENE | 32.86 |
| 135TMBENZENE | 10.09 |
| INDANE | 0.78 |
| OTHER C10 PARAFFINS | |
| 1M3NC3BENZENE | 1.06 |
| 1M4NC3BENZENE | 0.42 |
| 1M3IC3BENZENE | |
| 1M4IC3BENZENE | 0.16 |
| 12DETBENZENE | 0.09 |
| 13DETBENZENE | 0.58 |
| 14DETBENZENE | 0.20 |
| 12DM3ETBENZENE | 0.24 |
| 12DM4ETBENZENE | 0.93 |
| 13DM2ETBENZENE | 0.07 |
| 13DM4ETBENZENE | 1.62 |
| 13DM5ETBENZENE | 2.05 |
| 14DM2ETBENZENE | 1.07 |
| 1234TMBENZENE | 0.14 |
| 1235TMBENZENE | 0.81 |
| 1245TMBENZENE | 0.64 |
| NAPHTHALENE | 0.05 |
| M-INDANES | 0.09 |
| OTHER C10 AROMATICS | 0.59 |
| 1M-NAPHTHALENE | 0.00 |
| 2M-NAPHTHALENE | 0.00 |
| OTHER C11 AROMATICS | 0.01 |
| Total | 100.0 |

### EXAMPLE 1 (COMPARATIVE)

A two bed stacked catalyst system was produced as follows:
A first (top) catalyst bed comprising Pt/Sn on steamed 50/50 ZSM-5/Silica.
A second (bottom) catalyst bed comprising Pt/Sn on 65/35 ZSM-12/Alumina.

The first catalyst was produced by steaming an H-form extrudate composed of 50 wt. % ZSM-5 (average crystal size (primary particle) of less than 0.05 microns) and 50 wt. % silica binder for 6 hours at 800° F. (427° C.) in 100% steam. 0.05 wt. % Pt as tetraammonium platinum nitrate and 0.12 wt. % Sn as Sn(II) chloride salt were co-impregnated via incipient wetness onto the steamed extrudate. The resultant catalyst was calcined in air at 680° F. (360° C.) for 3 hours.

The second catalyst was produced by co-impregnating an H-form extrudate of 65 wt. % ZSM-12 and 35 wt. % alumina binder via incipient wetness with 0.05 wt. % Pt as tetraammonium platinum nitrate and 0.12 wt. % Sn as Sn(II) chloride salt. The catalyst was then calcined in air at 680° F. (360° C.) for 3 hours.

The catalyst particles were targeted to a length/diameter ratio of about 1. The particles were loaded into a fixed bed reactor so as to produce a stacked bed arrangement comprising 1 gm of the first catalyst as the top bed and 2.4 gm of the second catalysts as the bottom bed. 80×120 mesh sand was used to fill any void spaces. The stacked bed catalyst was heated in hydrogen and activated at 400° C. for 1 hour. The temperature was then increased to 430° C. and liquid feed from Table 1 was introduced in down flow mode so as to initially contact the top bed for a 12 hour de-edging period. De-edging conditions were as follows:
WHSV: 4.4
H₂/HC molar ratio: 1
Pressure: 350 psig

Following de-edging, conditions were modified and a temperature scan was performed under the following conditions:
WHSV: 4.4
H₂/HC molar ratio: 1
Temperature 12 hours at 390° C., 400° C., 410° C., 420° C., and a return to 380° C.
Pressure: 350 psig

On-line GC analyses were performed every 4 hours using a front detector with a DB1 column to speciate light ends and a back detector with a DBWax column for the heavier products.

### EXAMPLE 2

The process of Example 1 was repeated with the second (bottom) catalyst bed comprising Pt/Sn on 32.5/32.5/35 ZSM-12/ZSM-5/Alumina. The second catalyst was produced by co-impregnating an H-form extrudate of 32.5 wt. % ZSM-12, 32.5 wt. % ZSM-5 (average crystal size (primary particle) of less than 0.05 microns), and 35 wt. % alumina binder via incipient wetness with 0.05 wt. % Pt as tetraammonium platinum nitrate and 0.12 wt. % Sn as Sn(II) chloride salt. The resultant catalyst was calcined in air at 680° F. (360° C.) for 3 hours.

### EXAMPLE 3

The process of Example 1 was repeated with the second (bottom) catalyst bed comprising Pt/Sn on 52/13/35 ZSM-12/ZSM-5/Alumina. The second catalyst was produced by co-impregnating an H-form extrudate of 52 wt. % ZSM-12, 13 wt. % ZSM-5 (average crystal size (primary particle) of less than 0.05 microns), and 35 wt. % alumina binder via incipient wetness with 0.05 wt. % Pt as tetraammonium platinum nitrate and 0.12 wt. % Sn as Sn(II) chloride salt. The resultant catalyst was calcined in air at 680° F. (360° C.) for 3 hours.

### EXAMPLE 4

The process of Example 1 was repeated with the second (bottom) catalyst bed comprising Pt/Sn on 65/15/20 ZSM-12/ZSM-5/Alumina. The second catalyst was produced by co-impregnating an H-form extrudate of 65 wt. % ZSM-12, 15 wt. % ZSM-5 (average crystal size (primary particle) of less than 0.05 microns), and 20 wt. % alumina binder via incipient wetness with 0.05 wt. % Pt as tetraammonium platinum nitrate and 0.12 wt. % Sn as Sn(II) chloride salt. The resultant catalyst was calcined in air at 680° F. (360° C.) for 3 hours.

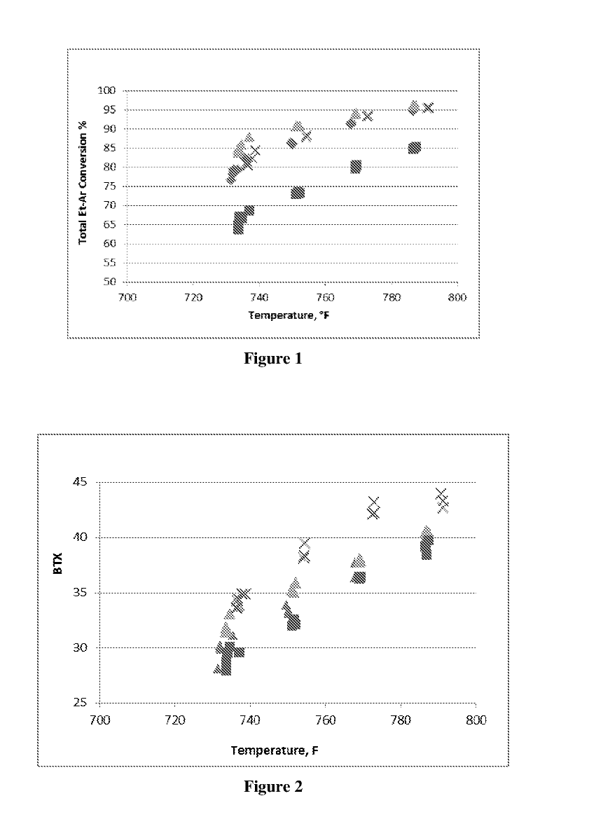

A comparison of the results of testing of the catalysts of Examples 1 to 4 is shown in FIGS. 1 and 2, in which the square-shaped points indicate the results obtained with the catalyst of Example 1, the triangular-shaped points indicate the results obtained with the catalyst of Example 2, the diamond-shaped points indicate the results obtained with the catalyst of Example 3 and the cross-shaped points indicate the results obtained with the catalyst of Example 4.

It will be seen from FIG. 1, which plots ethyl aromatic conversion against temperature, that the addition of ZSM-5 to the second catalyst greatly enhances the de-alkylation activity of the overall catalyst system. In particular, FIG. 1 shows that the catalyst systems of Examples 2 to 4 achieve equivalent ethyl-aromatic conversion at much lower temperatures than the catalyst system of Example 1. While the de-alkylation activity is high for all formulations of Examples 2 to 4, it will be seen from FIG. 2, which plots BTX yield against temperature, that the amount of ZSM-12, ZSM-5, and total zeolite in the co-extrudate also impacts product yields. In particular, it will be seen that increased ZSM-12 content improves the benzene/toluene/xylene yields, with the catalyst system of Example 4 with 65% ZSM-12 and 15% ZSM-5 showing the highest BTX yield of all formulations.

### EXAMPLE 5

The process of Example 4 was repeated with the 65/15/20 ZSM-12/ZSM-5/Alumina extrudate being steamed for 4 hours at 800° F. (427° C.) in 100% steam at atmospheric pressure before co-impregnation with 0.05 wt. % Pt as tetraammonium platinum nitrate and 0.12 wt. % Sn as Sn(II) chloride salt. The resultant catalyst was calcined in air at 680° F. (360° C.) for 3 hours.

### EXAMPLE 6

The process of Example 4 was repeated with the 65/15/20 ZSM-12/ZSM-5/Alumina extrudate being steamed for 5.25 hours at 900° F. (482° C.) in 100% steam at atmospheric pressure before co-impregnation with 0.05 wt. % Pt as tetraammonium platinum nitrate and 0.12 wt. % Sn as Sn(II) chloride salt. The resultant catalyst was calcined in air at 680° F. (360° C.) for 3 hours.

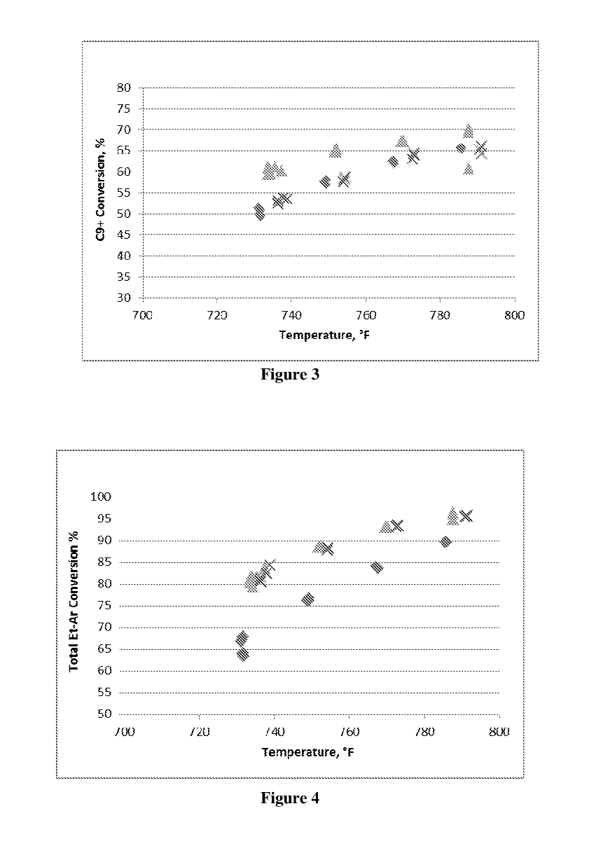

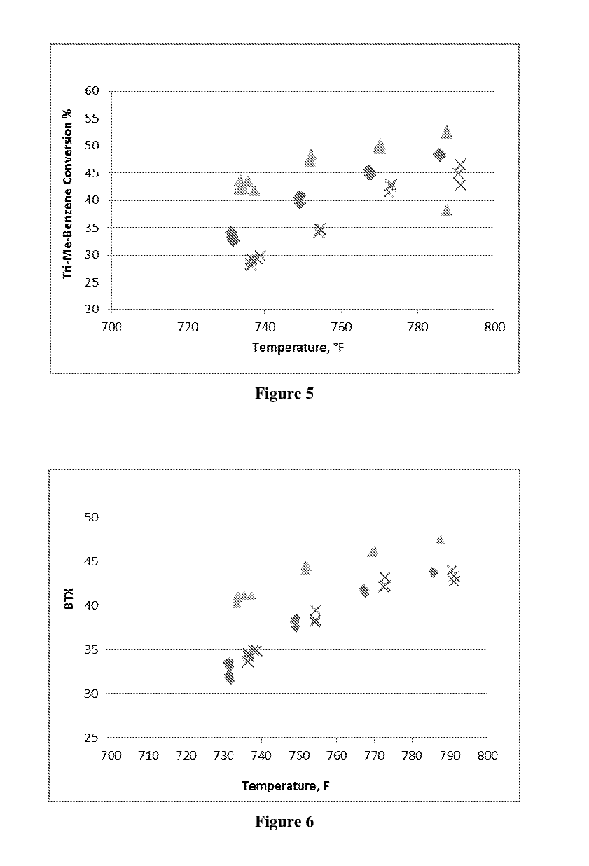

A comparison of the results of testing of the catalysts of Examples 4 to 6 is shown in FIGS. 3 to 6, in which the cross-shaped points indicate the results obtained with the catalyst of Example 4, the triangular-shaped points indicate the results obtained with the catalyst of Example 5 and the diamond-shaped points indicate the results obtained with the catalyst of Example 6. In particular, FIGS. 3 to 5 compare the overall C₉₊ conversion, the ethyl-aromatic conversion and the trimethylbenzene conversion of the unsteamed catalyst of Example 4 and the steamed catalysts of Examples 5 and 6, while FIG. 6 compares the BTX yield against temperature for the same catalysts. When steaming conditions are adjusted appropriately, as in Example 5, overall catalyst activity is increased as shown by the increase in C₉₊ conversion vs. temperature. Interestingly, the de-ethylation activity of the moderately steam catalyst of Example 5 is similar to the unsteamed version of Example 4, indicating that transalkylation activity has been impacted. BTX yields are also very high for the moderately steam catalyst of Example 5. More severely steaming the catalyst, as in Example 6, decreases the de-ethylation activity but the transalkylation activity is improved as evidenced by improved TMB conversion in FIG. 5. BTX yields look comparable between the unsteamed and severely steamed versions (see FIG. 6). This work indicates that catalyst performance can be optimized based on steaming conditions.

### EXAMPLE 7

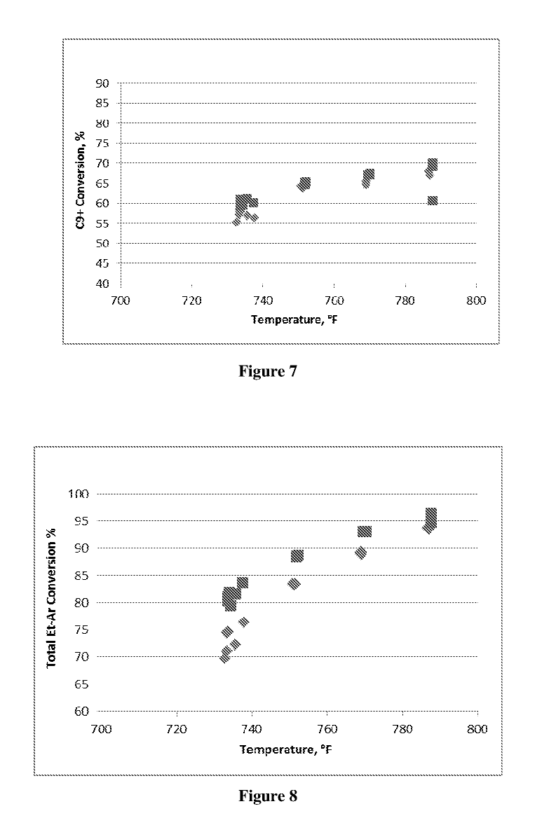

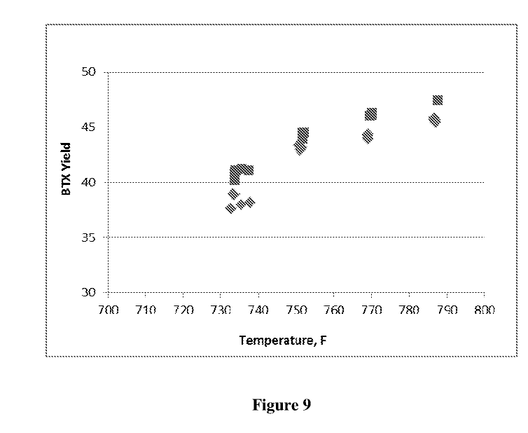

A single bed catalyst system was evaluated using the Pt/Sn steamed (4 h at 427° C.) /65/15/20 ZSM-12/ZSM-5B/Alumina second bed catalyst of Example 5 as the single catalyst bed. The resultant catalyst system was subjected to the de-edging and temperature scanning procedures of Example 1 and the results are compared with those obtained in Example 5 in FIGS. 7 to 9. In FIGS. 7 to 9, the square-shaped points indicate the results obtained with the stacked bed catalyst of Example 5 and the diamond-shaped points indicate the results obtained with the single bed catalyst of Example 7. Overall activity of the two catalysts systems was similar as shown by C₉₊ conversion vs. temperature in FIG. 7. However, the de-ethylation activity was higher for the stacked bed as shown in FIG. 8. Furthermore, BTX yields were improved for the stacked bed configuration as shown in FIG. 9.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

All documents described herein are incorporated by reference herein, including any priority documents and/or testing procedures to the extent they are not inconsistent with this text. As is apparent from the foregoing general description and the specific embodiments, while forms of the invention have been illustrated and described, various modifications can be made without departing from the spirit and scope of the invention. Accordingly, it is not intended that the invention be limited thereby. Likewise, the term “comprising” is considered synonymous with the term “including.” Likewise whenever a composition, an element or a group of elements is preceded with the transitional phrase “comprising”, it is understood that we also contemplate the same composition or group of elements with transitional phrases “consisting essentially of,” “consisting of”, “selected from the group of consisting of,” or “is” preceding the recitation of the composition, element, or elements and vice versa.

1. A process for producing xylene from C₉₊ aromatic hydrocarbons, the process comprising:
(a) contacting a first feed comprising C₉₊ aromatic hydrocarbons and hydrogen with a first catalyst composition under conditions effective to dealkylate at least part of the C₉₊ aromatic hydrocarbons containing C₂₊ alkyl groups and to saturate the resulting C₂₊ olefins to produce a second feed, wherein the first catalyst composition comprises a first molecular sieve having a Constraint Index of 3 to 12 and at least one hydrogenation component; and
(b) contacting the second feed with a second catalyst composition under conditions effective to transalkylate at least part of the C₉₊ aromatic hydrocarbons in the second feed to produce a first product comprising xylene, wherein the second catalyst composition comprises a second molecular sieve having a Constraint Index less than 3 and a third molecular sieve having a Constraint Index of 3 to 12.

2. The process of claim 1, wherein the first feed further comprises at least one C₆-C₇ aromatic hydrocarbon.

3. The process of claim 1, wherein the first molecular sieve comprises ZSM-5.

4. The process of claim 1, wherein the first molecular sieve comprises ZSM-5 crystals having an external surface area in excess of 50 m²/g as determined by the t-plot method for nitrogen physisorption.

5. The process of claim 1, wherein the at least one hydrogenation component of the first catalyst composition comprises at least one metal or compound thereof selected from Groups 6-10 of the Periodic Table and tin or a tin compound.

6. The process of claim 1, wherein the second molecular sieve comprises ZSM-12.

7. The process of claim 1, wherein the second molecular sieve comprises mordenite.

8. The process of claim 1, wherein the third molecular sieve comprises ZSM-5.

9. The process of claim 1, wherein the second molecular sieve and the third molecular sieve are contained in a single catalyst particle.

10. The process of claim 9, wherein the single catalyst particle is produced by co-extrusion.

11. The process of claim 1, wherein the second catalyst composition comprises from 50 wt. % to 85 wt. % of the second molecular sieve and from 15 wt. % to 50 wt. % of the third molecular sieve based on the total weight of the second and third molecular sieves.

12. The process of claim 1, wherein at least the molecular sieve components of the second catalyst composition are presteamed with from 5 to 100% steam at a temperature from 260 to 650° C. for at least one hour.

13. The process of claim 1, wherein the second catalyst composition further comprises at least one hydrogenation component.

14. The process of claim 13, wherein the at least one hydrogenation component of the second catalyst composition comprises at least one metal or compound thereof selected from Groups 6-10 of the Periodic Table and tin or a tin compound.

15. The process of claim 1 and further comprising:
(c) contacting at least part of the first product with a third catalyst composition under conditions effective to remove benzene coboilers in the first product and produce a second product, wherein the third catalyst composition comprises a fourth molecular sieve having a Constraint Index of 3 to 12; and
(d) recovering xylene from the second product.

16. The process of claim 15, wherein the fourth molecular sieve comprises ZSM-5.

17. The process of claim 15, wherein the fourth molecular sieve comprises ZSM-5 crystals having an external surface area in excess of 50 m²/g as determined by the t-plot method for nitrogen physisorption.

18. A catalyst system for transalkylating a feed comprising C₉₊ aromatic hydrocarbons to produce xylene, the catalyst system comprising:
(i) a first catalyst composition comprising a first molecular sieve having a Constraint Index of 3 to 12 and a hydrogenation component;
(ii) a second catalyst composition comprising a second molecular sieve having a Constraint Index less than 3 and a third molecular sieve having a Constraint Index of 3 to 12, the second catalyst composition being located downstream of the first catalyst composition when the catalyst system is contacted with the feed; and
(iii) a third catalyst composition comprising a fourth molecular sieve having a Constraint Index of 3 to 12, the third catalyst composition being located downstream of the second catalyst composition when the catalyst system is contacted with the feed.

19. The catalyst system of claim 18 and comprising from 15 wt. % to 35 wt. % of the first catalyst composition, from 50 wt. % to 75 wt. % of the second catalyst composition, and from 5 wt. % to 25 wt. % of the third catalyst composition, based on the total weight of the first, second and third catalyst compositions.
